(19) United States
(12) Patent Application Publication
(10) Pub. No.: US 2017/0363629 A1
(43) Pub. Date: Dec. 21, 2017

(54) BIOMARKERS AND TARGETS FOR CANCER IMMUNOTHERAPY

(71) Applicant: Board of Regents, The University of Texas System, Austin, TX (US)

(72) Inventors: Laszlo RADVANYI, Lutz, FL (US); Jie Qing CHEN, Tampa, FL (US); Patrick HWU, Houston, TX (US)

(21) Appl. No.: 15/524,605

(22) PCT Filed: Nov. 5, 2015
(86) PCT No.: PCT/US15/59284

§ 371 Date: May 4, 2017

Related U.S. Application Data
(60) Provisional application No. 62/075,603, filed on Nov. 5, 2014.

(51) Int. Cl. G01N 33/574; A61K 35/17; C12Q 1/68

(52) U.S. Cl. CPC G01N 33/5743; C12Q 1/6886; A61K 35/17; C12Q 2600/158; G01N 2800/52; C12Q 2600/106

(57) ABSTRACT

Provided herein are predictive biomarker signatures that identify patients as likely to benefit from TIL therapy. Also provided are tumor immunotherapy resistance pathways that may be targets of combination therapies to enhance TIL therapy.

The present application claims the priority benefit of U.S. provisional application No. 62/075,603, filed Nov. 5, 2014, the entire contents of which is incorporated herein by reference.

### BACKGROUND OF THE INVENTION

### 1. Field of the Invention

The present invention relates generally to the fields of cancer biology and immunology. More particularly, it concerns methods of predicting the response of a cancer patient to immunotherapy, such as expanded autologous tumor-infiltrating lymphocytes.

### 2. Description of Related Art

Adoptive cell therapy using expanded autologous tumor-infiltrating lymphocytes (TIL) is a promising immunotherapy for metastatic melanoma (Radvanyi et al., 2012). However, relatively little is known about the types of T cells in TIL mediating tumor regression, and no biomarker studies have been performed on the actual tumors used to expand TIL to identify factors predictive of clinical response. Thus, one of the critical areas in need of development to facilitate the further dissemination of T-cell and other immunotherapies is the discovery of specific biomarkers predicting who will respond to immunotherapy and who will develop resistance.

### SUMMARY OF THE INVENTION

Provided herein are tumor gene biomarkers (LTF and IRAK-1) that can be used to predict, either individually or in combination as a biomarker signature, who will have a positive clinical response and improved overall survival and progression-free survival in response to T-cell adoptive cell therapy using autologous tumor-infiltrating lymphocytes (TIL) for metastatic melanoma. These biomarkers can also be applied to other forms of immunotherapy for melanoma as well as immunotherapy for other forms of cancer.

In one embodiment, a method is provided for treating a patient having cancer comprising administering an effective amount of an anti-cancer immunotherapy to the patient, said patient having been determined to have a cancer comprising an elevated expression level of LTF compared to a reference expression level and/or a decreased expression level of IRAK-1 compared to a reference expression level. For example, in some aspects, the anti-cancer immunotherapy is a therapy comprising administration of an immunogenic composition including cancer cell antigen (e.g., a cancer vaccine), a cytokine, an antibody that activates the immune system (e.g., an anti-PD-1 or anti-CTLA-4 antibody), an antigen presenting cell (that stimulates immune effector cell production) or administration of immune effector cells themselves (e.g., autologous or allogeneic immune effector cells). For example, the immune effector cells can be T-cells, NK cells, NK T-cells, or precursors of these cells. In further aspects, immune effector cells for use according to the embodiments are engineered immune effector cells, such as cells comprising a transgene encoding a T-cell receptor (TCR) or chimeric antigen receptor (CAR). In certain aspects, a method is provided for treating a patient having cancer comprising administering an effective amount of an autologous tumor-infiltrating lymphocytes to the patient, said patient having been determined to have a cancer comprising an elevated expression level of LTF compared to a reference expression level and/or a decreased expression level of IRAK-1 compared to a reference expression level.

In one aspect, the patient may have been determined to have a cancer comprising an elevated expression level of LTF compared to a reference expression level. In one aspect, the patient may have been determined to have a cancer comprising a decreased expression level of IRAK-1 compared to a reference expression level. In various aspects, the patient may have been determined to have a cancer comprising an elevated expression level of LTF compared to a reference expression level and a decreased expression level of IRAK-1 compared to a reference expression level. In certain aspects, the reference expression level may be an expression level in a sample of healthy tissue.

In some aspects, the cancer may be melanoma, such as metastatic melanoma.

In various aspects, the level of LTF and/or IRAK-1 may be a protein level. In some aspects, the protein level may be determined by mass spectrometry, ELISA, flow cytometry, immunohistochemistry, western blot, radioimmunoassay, or immunoprecipitation. In various aspects, the level of LTF and/or IRAK-1 may be an mRNA level. In some aspects, the mRNA level may be determined by an array hybridization, direct hybridization of RNA, digital quantitation of transcript levels, quantitative PCR, quantitative sequencing, or northern blot assay.

In certain aspects, the method may further comprise administering a second anticancer therapy (e.g., in combination with an immunotherapy). In some aspects, the second anticancer therapy may be an anti-LTF therapy or a therapy with a purified or recombinant LTF polypeptide. In further aspects, the second anti-cancer therapy is an IRAK-1 inhibitor therapy. For example, the IRAK-1 inhibitor for use in a therapy can be a small molecule IRAK-1 inhibitor, such as 1-(2-(4-Morpholinyl)ethyl)-2-(3-nitrobenzoylamino)benzimidazole, N-(2-Morpholinylethyl)-2-(3-nitrobenzoylamido)-benzimidazole. In various aspects, the second anticancer therapy may be a surgical therapy, chemotherapy, radiation therapy, cryotherapy, hormonal therapy, toxin therapy, immunotherapy, or cytokine therapy.

In one embodiment, a method of treating a cancer patient is provided, the method comprising administering an effective amount of an anti-LTF therapy in combination with an immunotherapy (e.g., an autologous TIL therapy) to the patient, said patient having been determined to have a cancer comprising an elevated expression level of LTF compared to a reference level. In a further embodiment there is provided a method of treating a cancer patient comprising administering an effective amount of an immunotherapy (e.g., an autologous TIL therapy) in combination with a LTF polypeptide (or a nucleic acid expression vector for LTF). For example, the immunotherapy can be administered before, after or essentially simultaneously with the LTF polypeptide (or a nucleic acid expression vector for LTF). In soma aspects, a LTF polypeptide for use according to the embodiments is a human LTF polypeptide, such as a polypeptide that has been produced recombinantly. In further aspects, the LTF polypeptide is a purified LTF polypeptide, such as a purified bovine, ovine or goat LTF polypeptide.

In one embodiment, a method is provided for treating a patient having cancer comprising administering an effective amount of autologous tumor-infiltrating lymphocytes to the patient, said patient having been determined to have a cancer that does not comprise an elevated level of nitrotyrosine compared to a reference level.

In one embodiment, a method is provided for identifying a cancer patient as a candidate for autologous TIL therapy comprising determining an expression level of LTF and/or IRAK-1 in the cancer, wherein an increased expression level of LTF compared to a reference expression level and/or a decreased expression level of IRAK-1 compared to a reference expression level is indicative of the cancer patient being a candidate for autologous TIL therapy.

In some aspects, the method may further comprise measuring the expression level of LTF and/or IRAK-1 in at least one reference sample. In certain aspects, the reference sample may be a sample of healthy tissue from the patient. In other aspects, the reference sample may be a sample from a healthy subject.

In various aspects, determining an expression level of LTF and/or IRAK-1 in the cancer may comprise measuring the expression level of LTF and/or IRAK-1 in the cancer, measuring an expression level of LTF and/or IRAK-1 in the reference sample, and comparing the amount of LTF and/or IRAK-1 in the cancer and the reference sample. In some aspects, the expression level may be a protein level. In some aspects, the expression level may be an mRNA level.

In various aspects, the method may further comprise reporting whether the cancer patient is a candidate for autologous TIL therapy. In some aspects, the reporting may comprise providing a written or electronic report. In certain aspects, the reporting may comprise providing a report to the patient, a healthcare worker, or a payee.

In one embodiment, a method is provided for characterizing a cancer comprising selectively testing a cancer sample to determine the level of expression of LTF and/or IRAK-1. In some aspects, the method may further comprise obtaining a sample of the cancer from a cancer patient. In certain aspects, an elevated expression level of LTF compared to a reference expression level and/or a decreased expression level of IRAK-1 compared to a reference expression level may indicate that autologous TIL can be expanded from the cancer. In some aspects, the method may further comprise identifying the cancer patient as being eligible for autologous TIL therapy. In some aspects, the method may further comprise administering autologous TIL to the patient.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the frequency of CD8+ T cells in peritumoral regions vs. frequency of CD8+ in TIL infused into TIL-treated patients as determined by immunohistochemistry. Left panel shows the percent CD8 T cells in the infused TIL; right panel shows the number of CD8 T cells in the infused TIL.

FIGS. 2A-D show the significant difference between the frequency of CD8+, CD4+, and CD3+ T cells in tumors that yielded TIL and those that did not. FIGS. 2A-B show the percentage of CD8+, CD4+, and CD3+ T cells seen in tumors from which TIL were successfully expanded. FIGS. 2C-D show the fraction of TILs (as a percentage of total cells) in tumors from which TIL were successfully expanded versus those from which TIL were not successfully expanded using QuanTILfy™.

FIG. 3 shows how the expression of Nitrotyrosine, shown as a Nitrotyrosine (NT) Score, as determined by immunohistochemistry together with CD8, CD4, and CD3 expression, is negatively associated with the percentage of CD8+, CD4+, and CD3+ T cells in tumors from which TIL were attempted to be expanded for therapy. The plot show that as NT Score increases, CD8+, CD4+, and CD3+ expression (TILs in the tumor) shows a downward trend. The square symbols show NT Score in tumors of patients who did not successfully expand TIL for therapy (TIL not grower), while the filled circles show NT Scores of tumors of patients who successfully expanded TILs for therapy (TIL grower).

FIG. 4 shows the significant (P<0.05) fold changes (Log 2) in gene expression between TIL therapy responders and not responders. NanoString nCounter™ gene expression analysis was performed on RNA extracted from formalin fixed paraffin embedded (FFPE) samples of tumor used to initially expand TIL for adoptive T-cell therapy from >50 metastatic melanoma patients receiving TIL therapy.

FIGS. 5A-D show the correlation of immunosuppressive pathway FoxP3, PD-L1, PD1, and IDO expression with CD8+ cell infiltration by NanoString nCounter™ analysis (FIG. 5A) and immunohistochemistry (FIGS. 5B-C). FIG. 5D shows the Kaplan-Meier analysis of overall survival (OS) in TIL-treated patients was examined.

FIGS. 6A-E show that LTF and IRAK1 expression differentiates between TIL therapy responders and non-responders. FIG. 6A shows the NanoString nCounter™ gene expression analysis of LTF and IRAK-1 between responder and non-responder patients (N=35) treated with TIL. FIG. 6B shows the receiver operating curve (ROC) analysis of combined LTF and IRAK-1 gene expression in predicting the response to TIL therapy. FIG. 6C shows the IHC scores for all TIL-treated patient samples by responder (PR/CR) versus non-responder (PD/SD) (P=0.006; Kruskal-Wallis test). FIG. 6D shows the log-rank tests and Kaplan-Meier survival analyses of OS from the date of receiving TIL treatment (P=0.0003, top-left), progression-free survival (PFS) from the date of receiving TIL treatment (P=0.0028, top-right), OS from the date of surgery to remove the tumor for expansion of TIL (P=0.0016, bottom-left), and OS from the date of first diagnosis (P=0.0182, bottom-right) in patients with LTF greater than or less than the median expression (0.72 LTF score). Top lines are >0.72; Bottom lines are ≦0.72. FIG. 6E shows the log-rank test and Kaplan-Meier survival analyses of OS from the date of receiving TIL treatment (P=0.2855, top-left), PFS from the date of receiving TIL treatment (P=0.0489, top-right), OS from the date of surgery to remove the tumor for expansion of TIL (P=0.3293, bottom-left), and OS from the date of first diagnosis (P=0.8907, bottom-right) in patients with IRAK-1 greater than or less than the median expression (30 IRAK-1 score). Top lines are ≦30; Bottom lines are >30.

### DESCRIPTION OF ILLUSTRATIVE EMBODIMENTS

The lactotransferrin (LTF) and interleukin-1 receptor activating kinase (IRAK-1) genes are provided herein as tumor specimen biomarkers that can be used to predict which patients will benefit from improved overall survival and progression-free survival in response to autologous TIL therapy. LTF is a member of the transferrin family capable of binding and transferring Fe³⁺ ions and plays various biological functions outside of its iron-binding role. IRAK-1 is a critical enzyme mediating the activation of NFκB in cells, thereby driving the release of inflammatory and pro-angiogenic cytokines in tumors. Higher expression of LTF in tumors, as found by both gene expression analysis and immunohistochemistry (IHC) staining and quantitation of protein expression in tumors, was associated with clinical benefit after immunotherapy, while higher IRAK-1 gene and protein expression using these methods was found to be associated with a lack of clinical benefit. LTF and IRAK-1 are also provided herein as targets of drug or immunomodulatory therapies to enhance T-cell therapy and other forms of immunotherapy for solid tumors (e.g., melanoma).

The present methods allow for the prediction of which patients will respond to cancer immunotherapy (e.g., TIL adoptive cell therapy), thereby providing for greatly improving response rates by selecting patients for TIL therapy and other cancer immunotherapies. Also contemplated are combination therapies manipulating these predictive gene products to improve the effectiveness of TIL therapy, other T-cell therapies, as well as all other immunotherapies for melanoma and other solid tumors in which these predictive genes may play a role.

### I. BIOMARKERS

For the methods provided herein, the term biological samples refers to any biological sample obtained from an individual, including body fluids, body tissue, cells, or other sources known to those skilled in the art. Also, the terms “sample” and “biological sample” are used interchangeably herein. For example, a sample can be a tissue sample, such as a tumor tissue biopsy or resection. Other samples may include a thin layer cytological sample, a fine needle aspirate sample, a fresh frozen tissue sample, a paraffin embedded tissue sample, or an extract or processed sample produced from any of a peripheral blood sample. Body fluids, such as lymph, sera, whole fresh blood, peripheral blood mononuclear cells, frozen whole blood, plasma (including fresh or frozen), urine, saliva, semen, synovial fluid, and spinal fluid are also suitable as biological samples. Samples can further include breast tissue, renal tissue, colonic tissue, brain tissue, muscle tissue, synovial tissue, skin, hair follicle, bone marrow, and tumor tissue.

The biomarkers (also referred to herein as a “marker”) provided herein can be detected using any method known in the art.

A. LTF

LTF has been tested in pre-clinical murine cancer models as a monotherapy to facilitate anti-tumor immune responses. A human recombinant version of LTF called Talactoferrin™ (TLF) has been tested in a number of clinical trials as a monotherapy or in combination with chemotherapy in renal cell carcinoma, lung cancer (non-small cell lung cancer), colon cancer, and head and neck cancer. LTF has not been tested in combination with another immunotherapy, such as adoptive T-cell therapy of other immunomodulatory therapy, such as T-cell checkpoint blockade. LTF has not been previously identified previously as a theranostic biomarker for immunotherapy of cancer.

The amino acid sequence and the cDNA sequence of human LTF, also called GIG12, HEL110, and HLF2, are described in Genbank Accession Nos. NP_002334 and NP_001186078 (Protein), and Genbank Accession Nos. NM_002343 and NM_001199149 (mRNA sequence).

B. IRAK-1

IRAK1 regulates NFκB signaling and pro-inflammatory cytokine production associated with decreased anti-tumor adaptive immunity. IRAK-1 is a key driver, along with TRAF6, of cancer-inducing inflammation in mouse models of spontaneous cancer development. In addition, IRAK-1 and TRAF6 have been reported to be over-expressed in bone marrow cells of humans suffering from myelodisplastic syndrome (MDS), and IRAK-1 has been found to be one of the key drivers of MDS, thereby linking chronic inflammation to MDS development. IRAK-1 has not previously been reported to be a biomarker for cancer immunotherapy.

The amino acid sequence and the cDNA sequence of human IRAK-1 are described in Genbank Accession Nos. NP_001020413, NP_001020414, and NP_001560 (Protein), and Genbank Accession Nos. NM_001025242, NM_001025243, and NM_001569 (mRNA sequence).

### II. DETECTION METHODS

In certain embodiments, the method comprises the steps of obtaining a biological sample from a mammal to be tested; detecting the expression level of a LTF and/or IRAK-1 gene product in the sample. In one embodiment, the biological sample is a cell sample from a tumor in the mammal. As used herein the phrase “selectively measuring” refers to methods wherein only a finite number of protein or nucleic acid (e.g., mRNA) markers are measured rather than assaying essentially all proteins or nucleic acids in a sample. For example, in some aspects “selectively measuring” nucleic acid or protein markers can refer to measuring no more than 100, 75, 50, 25, 15, 10, 5, or 2 different nucleic acid or protein markers.

In one embodiment of the methods described herein, detecting the presence a gene product in a biological sample obtained from an individual comprises determining the level of an mRNA in the sample. The level of an mRNA in the sample can be assessed by combining oligonucleotide probes derived from the nucleotide sequence of the gene product to be detected with a nucleic acid sample from the individual, under conditions suitable for hybridization. Hybridization conditions can be selected such that the probes will hybridize only with the specified gene sequence. In one specific embodiment, conditions can be selected such that the probes will hybridize only with an altered nucleotide sequences, such as but not limited to, splice isoforms, and not with unaltered nucleotide sequences; that is, the probes can be designed to recognize only particular alterations in the nucleic acid sequence of the mRNA, including addition of one or more nucleotides, deletion of one or more nucleotides or change in one or more nucleotides (including substitution of a nucleotide for one which is normally present in the sequence). In one specific embodiment, the oligonucleotide probe hybridizes to the LTF mRNA sequence set forth as Genbank Deposit Nos. NM_002343 or NM_001199149, or to a coding region of the mRNA sequence, or to the IRAK-1 mRNA sequence set forth as Genbank Deposit Nos. NM_001025242, NM_001025243, or NM_001569, or to the coding region of a mRNA sequence.

Methods of quantitating mRNA in a sample are well-known in the art. In a particular embodiment, oligonucleotide probes specific to LTF and/or IRAK-1 can be displayed on an oligonucleotide array or used on a DNA chip. The term “microarray” refers to an array of distinct polynucleotides or oligonucleotides synthesized on a substrate, such as paper, nylon or other type of membrane, filter, chip, glass slide, or any other suitable solid support. Microarrays also include protein microarrays, such as protein microarrays spotted with antibodies. Another technique is directly measuring the levels (copy number) of LTF and/or IRAK-1 transcripts in isolated RNA from tumors or cells directly using a fluorescent DNA probe technology (direct digital readout of RNA transcript abundance). Other techniques for detecting LTF and/or IRAK-1 mRNA levels in a sample include reverse transcription of mRNA, followed by PCR amplification with primers specific for a LTF and/or IRAK-1 mRNA (e.g., RT-PCR or quantitative RT-PCR), in situ hybridization, Northern blotting, or nuclease protection.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR may be performed using a reference standard. The ideal reference standard is expressed at a constant level among different tissues, and is unaffected by the experimental treatment.

Suitable reference standards include, but are not limited to, mRNAs for the housekeeping genes glyceraldehyde-3-phosphate-dehydrogenase (GAPDH) and β-actin. The level of mRNA in the original sample or the fold change in expression of each biomarker may be determined using calculations well known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

In another embodiment of the methods described herein, detecting the presence a gene product in a biological sample obtained from an individual comprises determining the level of a polypeptide in the sample. The level of a gene product can be determined by contacting the sample with an antibody that specifically binds to the polypeptide product and determining the amount of bound antibody, e.g., by detecting or measuring the formation of the complex between the antibody and the polypeptide. The antibodies can be labeled (e.g., radioactive, fluorescently, biotinylated or HRP-conjugated) to facilitate detection of the complex. Appropriate assay systems for detecting polypeptide levels include, but are not limited to, flow cytometry, Enzyme-Linked Immunosorbent Assay (ELISA), competition ELISA assays, Radioimmuno-Assays (RIA), immunofluorescence, gel electrophoresis, Western blot, and chemiluminescent assays, bioluminescent assays, immunohistochemical assays that involve assaying a gene product in a sample using antibodies having specificity for the polypeptide product. Numerous methods and devices are well known to the skilled artisan for the detection and analysis of the instant invention. With regard to polypeptides or proteins in test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as but not limited to, biosensors and optical immunoassays, may be employed to determine the presence or amount of analytes without the need for a labeled molecule.

Alternatively, the level of a LTF and/or IRAK-1 polypeptide may be detected using mass spectrometric analysis. Mass spectrometric analysis has been used for the detection of proteins in serum samples. Mass spectroscopy methods include Surface Enhanced Laser Desorption Ionization (SELDI) mass spectrometry (MS), SELDI time-of-flight mass spectrometry (TOF-MS), Maldi Qq TOF, MS/MS, TOF-TOF, ESI-Q-TOF and ION-TRAP.

A polypeptide can be detected and quantified by any of a number of means known to those of skill in the art, including analytic biochemical methods, such as electrophoresis, capillary electrophoresis, high performance liquid chromatography (“HPLC”), thin layer chromatography (“TLC”), hyperdiffusion chromatography, and the like, or various immunological methods, such as fluid or gel precipitation reactions, immunodiffusion (single or double), immunoelectrophoresis, radioimmunoassay (“RIA”), enzyme-linked immunosorbent assay (“ELISA”), immunofluorescent assays, flow cytometry, FACS, western blotting, and the like.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye.

The labeled biomarker proteins are incubated with the antibody microarray. After washes to remove the unbound proteins, the microarray is scanned. The raw fluorescent intensity data may be converted into expression values using means known in the art.

### III. TREATMENT OF NEOPLASTIC CONDITIONS

The term “patient” means all mammals including humans. Examples of patients include humans, cows, dogs, cats, goats, sheep, pigs, and rabbits. Preferably, the patient is a human.

Autologous tumor-infiltrating lymphocytes (TIL) can be prepared and expanded according to any method known in the art, such as, for example, according to the methods described in U.S. Pat. No. 5,126,132 and PCT Publ. No. WO2012/129201, which are incorporated herein by reference in their entirety.

The methods described herein are useful in treating cancer, particularly, metastatic disease. Generally, the terms “cancer” and “cancerous” refer to or describe the physiological condition in mammals that is typically characterized by unregulated cell growth. More specifically, cancers that are treated in connection with the methods provided herein include, but are not limited to, carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, melanoma, superficial spreading melanoma, lentigo maligna melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted and monitored and/or patients benefiting from such treatments can be identified or selected via the methods described herein.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the treatment of disease, the appropriate dosage of an therapeutic composition will depend on the type of disease to be treated, as defined above, the severity and course of the disease, the patient's clinical history and response to the agent, and the discretion of the attending physician. The agent is suitably administered to the patient at one time or over a series of treatments.

### IV. COMBINATION TREATMENTS

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Autologous TIL may be administered before, during, after, or in various combinations relative to an anti-cancer treatment. The administrations may be in intervals ranging from concurrently to minutes to days to weeks. In embodiments where autologous TIL is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the autologous TIL and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below autologous TIL is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omega11); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorub ic in, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γrays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Predictive Immune Biomarker Signatures in The Tumor Microenvironment of Melanoma Metastases Associated With Tumor-Infiltrating Lymphocyte (TIL) Therapy

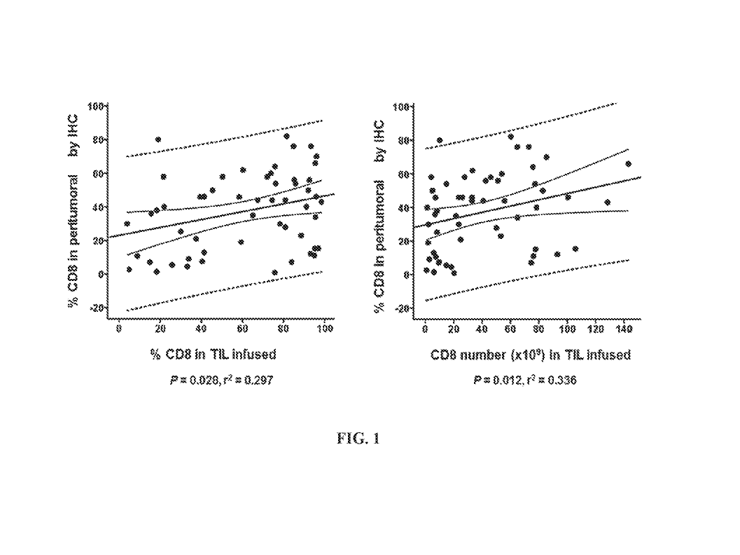

Frequency of CD8+ T cells in peritumoral regions vs. frequency of CD8+ in TIL infused into TIL-treated patients. Melanoma tumor samples were immunohistochemically stained for CD8, CD4, and CD3 and found to express CD8, CD4, and CD3. Further staining of melanoma tumor samples for CD8 revealed a significant association between the CD8 expression in the original tumors and the percentage of CD8+ T cells in final TIL product after expansion (FIG. 1).

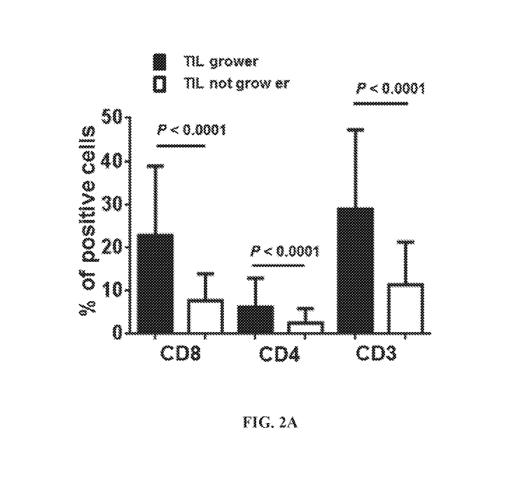

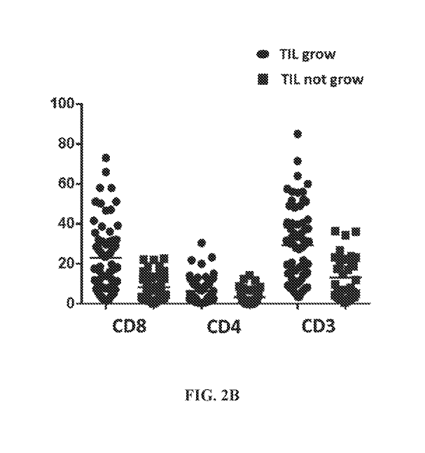

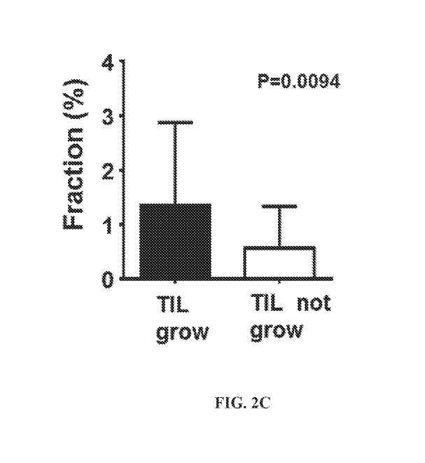

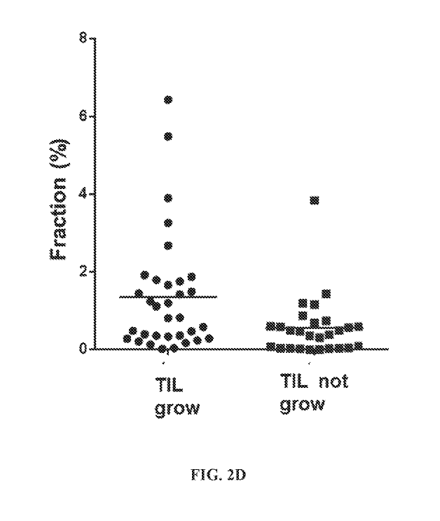

Significant difference between the frequency of CD8+, CD4+, and CD3+ T cells in tumors that yielded TIL and those that did not. The frequency of CD8+, CD4+, and CD3+ T cells in tumors from which TIL were successfully expanded (TIL grower) versus those from which TIL were not successfully expanded (TIL not grower) was determined by immunohistochemistry. A significantly increased percentage of CD8+, CD4+, and CD3+ T cells were seen in tumors from which TIL were successfully expanded (P<0.0001; FIGS. 2A-B).

Next, the fraction of TILs (as a percentage of total cells) was determined in tumors from which TIL were successfully expanded versus those from which TIL were not successfully expanded using a droplet digital PCR assay using TCR Vβ-specific primers called QuanTILfy™ (see U.S. Pat. Publ. No. 2014/0186848). The good TIL growers had a higher TCR Vβ gene signal (i.e. a larger fraction of TILs in the sample) than samples from poor growers (P=0.008; Mann-Whitney test) suggesting that this genetic test may be useful in selecting patients for TIL therapy (FIGS. 2C-D).

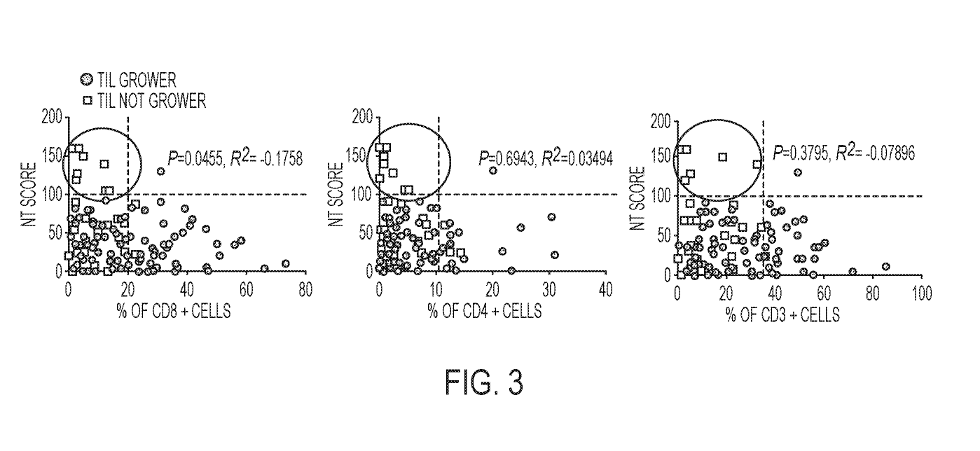

Nitrotyrosine (NT) Score together with CD8, CD4, and CD3 expression predict TIL grower status. Nitrotyrosine Score (NT Score), as determined by immunohistochemistry together with CD8, CD4, and CD3 expression, was found to be negatively associated with the percentage of CD8+, CD4+, and CD3+ T cells in tumors from which TIL were attempted to be expanded for therapy. NT is generated by the action of local peroxynitrite in the tumor or tissue or cells as a result of a spontaneous reaction between nitric oxide (NO) and reactive oxygen species (ROS), an occurrence that can happen in solid tumors. Peroxynitrite is very reactive and causes protein nitrosylation modifying tyrosine, tryptophan, and cysteine, and other amino acids that affect normal protein function. An antibody stain by IHC can measure NT levels, and an increased NT score reflects increased abnormal protein function, which can be, for example, defective a chemokine or chemokine receptor functioning negatively affecting the migration of T cells and other cells into tumors or other inflamed tissues. The plots (FIG. 3) show that increased NT Score is associated with a downward trend in the expression of CD8+, CD4+, and CD3+ expression (TILs in the tumor). Tumors from patients that have a NT Score of 150 or above were unsuccessful in TIL outgrowth for therapy, with one exception of a tumor from a patient with successful TIL expansion that had an NT Score above 150, as shown in FIG. 3. Thus, an NT Score above 150, or any comparable score, may be used as a predictor by itself or in combination with other markers of TIL content in tumors as well as the likelihood of successfully numerically expanding TIL from tumors for adoptive cell therapy.

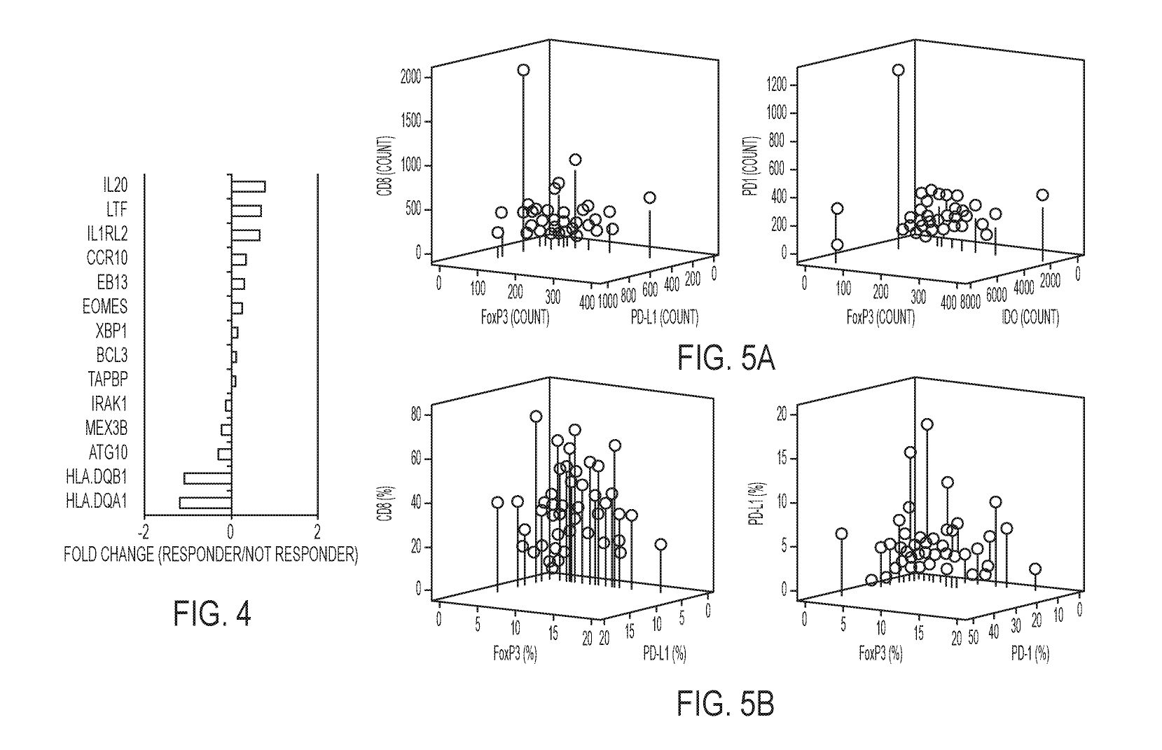

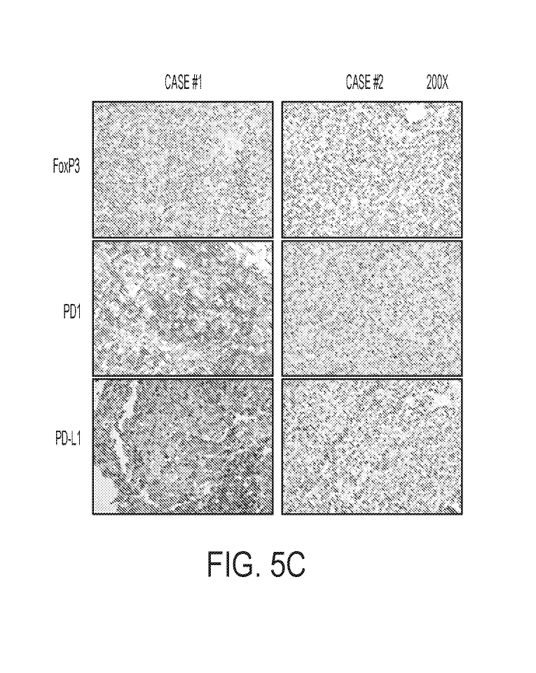

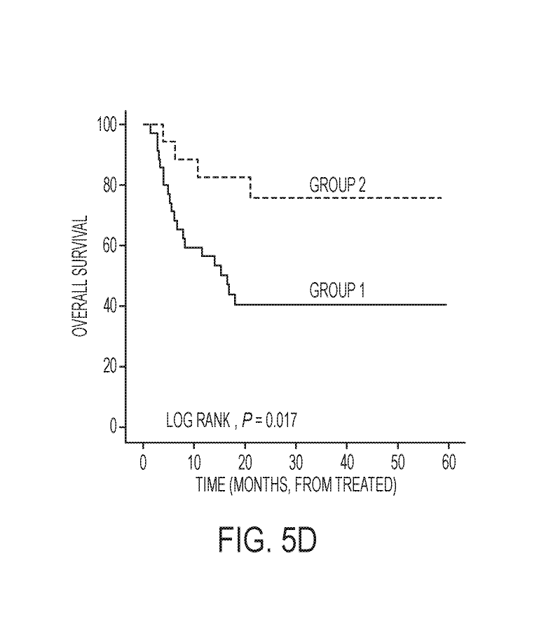

Hierarchical clustering of gene expression between TIL grow and TIL not grow. NanoString nCounter™ gene expression analysis of 595 immunologically-relevant genes was performed on RNA extracted from formalin fixed paraffin embedded (FFPE) samples of tumors used to initially expand TIL for adoptive T-cell therapy from >50 metastatic melanoma patients receiving TIL therapy (FIG. 4). Differences were found in a number of genes in tumors of patients having good TIL growth versus poor TIL growth, such as CD8β and CD3δ, CD45RA, ICOS, PD-1, and STAT4. CD8 gene expression was significantly correlated with immunosuppression pathway genes (FIG. 5A), such as PD-L1, PD-1, FoxP3, and IDO, which was confirmed by IHC analysis (FIGS. 5B-C). Kaplan-Meier analysis of overall survival (OS) in TIL-treated patients was examined. OS probably was evaluated (in months from TIL treatment) for combined CD8 and FoxP3 immunohistochemistry expression both intratumorally and peritumorally. The samples were divided into two groups for analysis: Group 1 had one or two high levels of expression among the four categories; Group 2 had three or four high levels of expression among the four categories (CD8 peritumoral; CD8 intratumoral; FoxP3 peritumoral; FoxP3 intratumoral). Group 2 was found to have significantly high overall survival (FIG. 5D). Thus, IHC analysis of these markers in original tumors together with CD8 was predictive of overall survival (OS) after TIL infusion

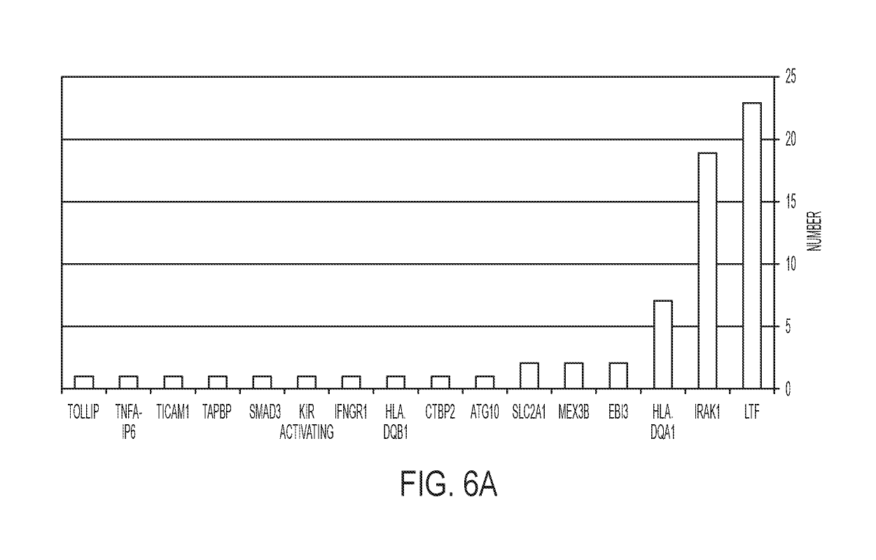

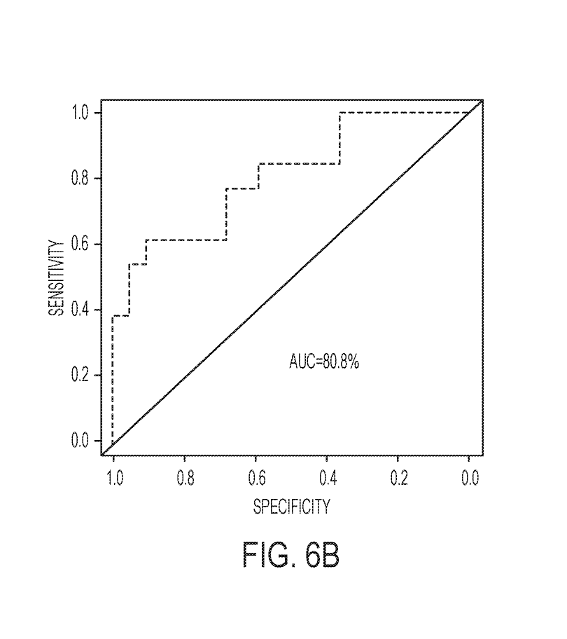

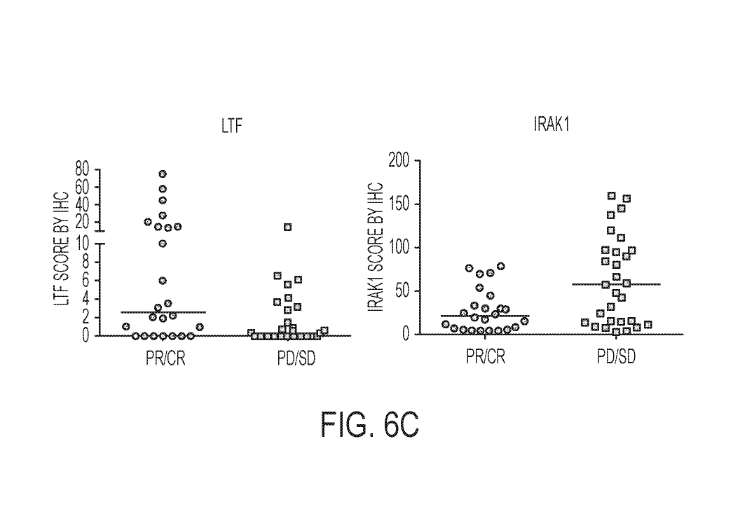

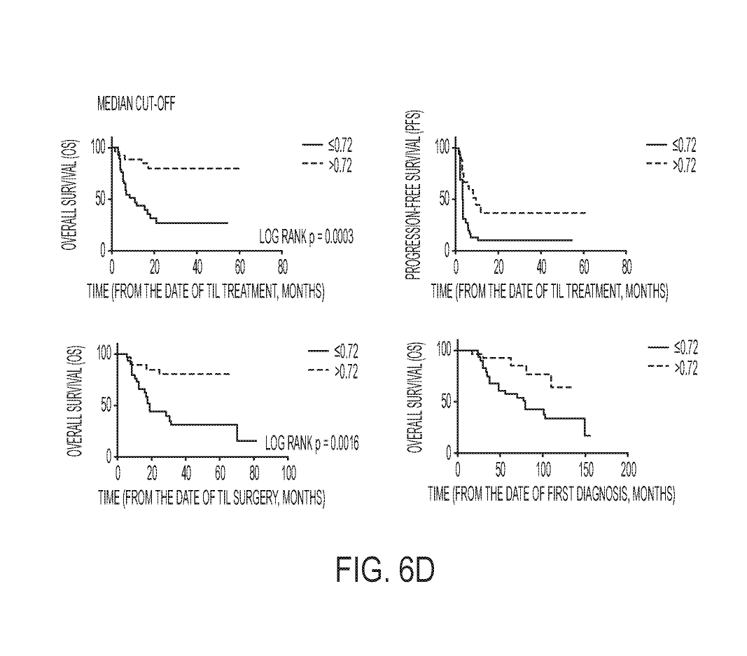

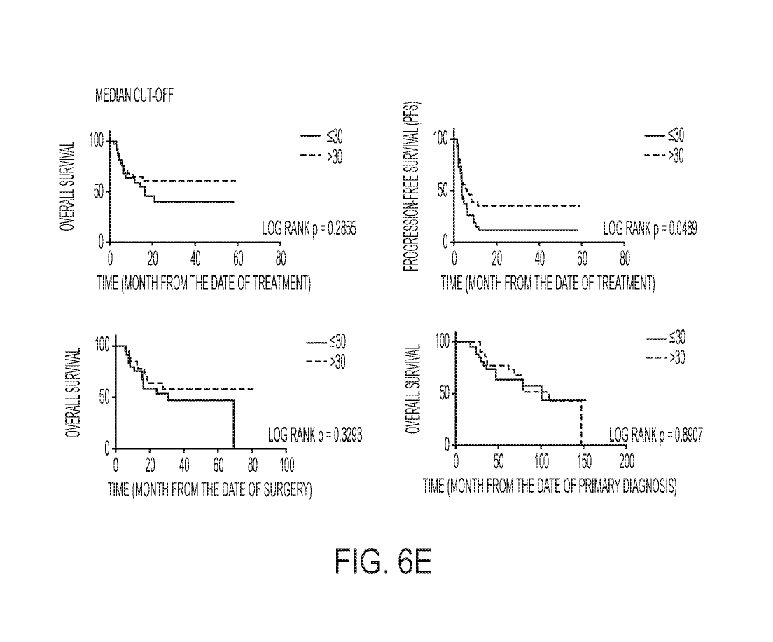

LTF and IRAK1 expression differentiates between TIL therapy responders and non-responders. The NanoString nCounter™ gene expression analysis revealed significant differences in LTF and IRAK-1 gene expression between responder and non-responder patients (N=35) treated with TIL. LTF and IRAK1 gene expression levels and IHC analyses were able to predict the response to TIL therapy with at least ten models in a leave-one-out logistic regression analysis between TIL therapy responders and non-responders (FIG. 6A), a powerful statistical test used to determine the robustness of a marker in a set of heterogeneous samples. LTF and IRAK-1 gene expression, when combined together, were also able predict the response to TIL therapy using receiver operating curve (ROC) analysis with an area under the curve of 80.8% (FIG. 6B). The IHC staining on all TIL-treated patient samples validated the mRNA data from NanoString nCounter™ analysis. Patients with higher LTF (FIG. 6C) and lower IRAK1 (FIG. 6C) protein expression by IHC responded to TIL therapy (P=0.006; Kruskal-Wallis test). Log-rank tests and Kaplan-Meier survival analyses revealed significantly longer OS (P=0.0003, FIG. 6D top-left) and progression-free survival (PFS) (P=0.0028, FIG. 6D top-right) in patients with LTF over the median expression (0.72 LTF score) from the date of receiving TIL treatment. An LTF IHC score above the median (0.72) also revealed a significantly longer OS from the date of surgery to remove the tumor for expansion of TIL (P=0.0016, FIG. 6D bottom-left) and OS from the date of first diagnosis in patients (P=0.0182, FIG. 6D bottom-right). Log-rank test and Kaplan-Meier survival analyses revealed significantly longer PFS (P=0.0489, FIG. 6E top-right) in patients with IRAK-1 less than the median expression (≦30) versus >30 from the date of receiving TIL treatment.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,126,132
U.S. Pat. Publ. No. 2014/0186848
PCT Publ. No. WO2012/129201
Galon et al., J. Transl. Med., 10:205, 2012.
Radvanyi et al., Clin. Cancer Res., 18:6758-6770, 2012.
Reis et al., BMC Biotechnology, 11:46, 2011.

What is claimed is:

1. A method of treating a patient having cancer comprising administering an effective amount of an anti-cancer immunotherapy to the patient, said patient having been determined to have a cancer comprising an elevated expression level of LTF compared to a reference expression level and/or a decreased expression level of IRAK-1 compared to a reference expression level.

2. A method of treating a patient having cancer comprising administering an effective amount of autologous tumor-infiltrating lymphocytes to the patient, said patient having been determined to have a cancer comprising an elevated expression level of LTF compared to a reference expression level and/or a decreased expression level of IRAK-1 compared to a reference expression level.

3. The method of claim 1 or 2, wherein said patient has been determined to have a cancer comprising an elevated expression level of LTF compared to a reference expression level.

4. The method of claim 1 or 2, wherein said patient has been determined to have a cancer comprising a decreased expression level of IRAK-1 compared to a reference expression level.

5. The method of claim 1 or 2, wherein said patient has been determined to have a cancer comprising an elevated expression level of LTF compared to a reference expression level and a decreased expression level of IRAK-1 compared to a reference expression level.

6. The method of claim 1 or 2, wherein the reference expression level is an expression level in a sample of healthy tissue.

7. The method of claim 1 or 2, wherein the cancer is melanoma.

8. The method of claim 7, wherein the melanoma is metastatic melanoma.

9. The method of claim 1 or 2, wherein the level of LTF and/or IRAK-1 is a protein level.

10. The method of claim 9, wherein the protein level is determined by mass spectrometry, ELISA, flow cytometry, immunohistochemistry, western blot, radioimmunoassay, or immunoprecipitation.

11. The method of claim 1 or 2, wherein the level of LTF and/or IRAK-1 is an mRNA level.

12. The method of claim 11, wherein the mRNA level is determined by an array hybridization, direct hybridization of RNA, digital quantitation of transcript levels, quantitative PCR, quantitative sequencing, or northern blot assay.

13. The method of claim 1, wherein the anti-cancer immunotherapy comprises administration of an immunogenic composition comprising a cancer cell antigen.

14. The method of claim 1, wherein the anti-cancer immunotherapy comprises administration of a cytokine or an antibody that activates the immune system.

15. The method of claim 14, wherein the antibody that activates the immune system is an anti-PD-1 or anti-CTLA-4 antibody.

16. The method of claim 1, wherein the anti-cancer immunotherapy comprises administration of an antigen presenting cell.

17. The method of claim 1, wherein the anti-cancer immunotherapy comprises administration of immune effector cells.

18. The method of claim 17, wherein the immune effector cells comprise T-cells targeted to a tumor antigen.

19. The method of claim 18, wherein the T-cells targeted to a tumor antigen comprise a transgene encoding a T-cell receptor or a chimeric antigen receptor.

20. The method of claim 1 or 2, further comprising administering a second anticancer therapy.

21. The method of claim 20, wherein the second anti-cancer therapy is an anti-LTF therapy.

22. The method of claim 20, wherein the second anticancer therapy comprises administration of a LTF polypeptide.

23. The method of claim 20, wherein the second anticancer therapy comprises administration of an IRAK-1 inhibitor.

24. The method of claim 20, wherein the second anticancer therapy is a surgical therapy, chemotherapy, radiation therapy, cryotherapy, hormonal therapy, toxin therapy, immunotherapy, or cytokine therapy.

25. A method of treating a cancer patient comprising administering an effective amount of an anti-LTF therapy in combination with an immunotherapy to the patient, said patient having been determined to have a cancer comprising an elevated expression level of LTF compared to a reference expression level.

26. The method of claim 25, wherein the immunotherapy is an autologous TIL therapy.

27. A method of identifying a cancer patient as a candidate for an anti-cancer immunotherapy comprising determining an expression level of LTF and/or IRAK-1 in the cancer, wherein an increased expression level of LTF compared to a reference expression level and/or a decreased expression level of IRAK-1 compared to a reference expression level is indicative of the cancer patient being a candidate for autologous TIL therapy.

28. A method of identifying a cancer patient as a candidate for autologous TIL therapy comprising determining an expression level of LTF and/or IRAK-1 in the cancer, wherein an increased expression level of LTF compared to a reference expression level and/or a decreased expression level of IRAK-1 compared to a reference expression level is indicative of the cancer patient being a candidate for autologous TIL therapy.

29. The method of claim 27 or 28, further comprising measuring the expression level of LTF and/or IRAK-1 in at least one reference sample.

30. The method of claim 29, wherein the reference sample is a sample of healthy tissue from the patient.

31. The method of claim 29, wherein the reference sample is a sample from a healthy subject.

32. The method of claim 29, wherein determining an expression level of LTF and/or IRAK-1 in the cancer comprises measuring the expression level of LTF and/or IRAK-1 in the cancer, measuring an expression level of LTF and/or IRAK-1 in the reference sample, and comparing the amount of LTF and/or IRAK-1 in the cancer and the reference sample.

33. The method of claim 32, wherein the expression level is a protein level.

34. The method of claim 32, wherein the expression level is an mRNA level.

35. The method of claim 27, wherein the anti-cancer immunotherapy comprises administration of an immunogenic composition comprising a cancer cell antigen.

36. The method of claim 27, wherein the anti-cancer immunotherapy comprises administration of a cytokine or an antibody that activates the immune system.

37. The method of claim 36, wherein the antibody that activates the immune system is an anti-PD-1 or anti-CTLA-4 antibody.

38. The method of claim 27, wherein the anti-cancer immunotherapy comprises administration of an antigen presenting cell.

39. The method of claim 27, wherein the anti-cancer immunotherapy comprises administration of immune effector cells.

40. The method of claim 39, wherein the immune effector cells comprise T-cells targeted to a tumor antigen.

41. The method of claim 40, wherein the T-cells targeted to a tumor antigen comprise a transgene encoding a T-cell receptor or a chimeric antigen receptor.

42. The method of claim 27, further comprising reporting whether the cancer patient is a candidate for an anti-cancer immunotherapy.

43. The method of claim 28, further comprising reporting whether the cancer patient is a candidate for autologous TIL therapy.

44. The method of claim 42, wherein the reporting comprises providing a written or electronic report.

45. The method of claim 42, wherein the reporting comprises providing a report to the patient, a healthcare worker, or a payee.

46. A method of characterizing a cancer comprising selectively testing a cancer sample to determine the level of expression of LTF and/or IRAK-1.

47. The method of claim 46, further comprising obtaining a sample of the cancer from a cancer patient.

48. The method of claim 46, wherein an elevated expression level of LTF compared to a reference expression level and/or a decreased expression level of IRAK-1 compared to a reference expression level indicates that autologous TIL can be expanded from the cancer.

49. The method of claim 48, further comprising identifying the cancer patient as being eligible for autologous TIL therapy.

50. The method of claim 49, further comprising administering autologous TIL to the patient.
